(19) United States
(12) Patent Application Publication
(10) Pub. No.: US 2025/0072979 A1
(43) Pub. Date: Mar. 6, 2025

(54) Predictive Maintenance for Robotically Assisted Surgical System

(71) Applicant: Auris Health, Inc., Redwood City, CA (US)

(72) Inventors: Anton Viktorovich Goldade, San Jose, CA (US); Matthew Kochman, Sunnyvale, CA (US)

(21) Appl. No.: 18/456,800

(22) Filed: Aug. 28, 2023

(51) Int. Cl. A61B 34/30; G06N 20/00; G16H 40/40

(52) U.S. Cl. CPC A61B 34/30; G06N 20/00; G16H 40/40

(57) ABSTRACT

A robotically assisted surgical system includes a robot and various control systems for facilitating assistance with a medical procedure. A predictive maintenance module obtains various operational data associated with the robot and applies a machine learning model trained to predict failures or degradations, classify a health state of the robot, and/or detect anomalous conditions that may be indicative of a future failure. The predictive maintenance module may invoke various actions in response to inferences generated by the machine learning model, such as generating notifications, generating messages to a connected software platform, and/or initiating automated actions associated with the operation of the robot.

### BACKGROUND

### Technical Field

The described embodiments relate to a system and a method for facilitating predictive maintenance activities in a robotically assisted surgical system.

### Description of the Related Art

Robotically assisted surgical systems may be employed to provide critical assistance for a variety of surgical procedures. Such systems may incorporate advanced imaging or other sensing capabilities and precision instrument control systems capable of facilitating complex medical procedures. Such systems may rely on various mechanical components, sensing devices, and control systems that may be prone to degrading or failing over time. Such degradations or failures can impact the scheduling and/or outcome of medical procedures.

### BRIEF DESCRIPTION OF THE DRAWINGS

FIG. 1 is an example embodiment of a medical environment for a robotically assisted surgical system.

FIG. 2 is a block diagram illustrating an example embodiment of a predictive maintenance module for a robotically assisted surgical system.

FIG. 3 is a flowchart illustrating an example embodiment of a process for operating a predictive maintenance module for a robotically assisted surgical system.

FIG. 4 is a visual representation of a technique for classifying operating states of a robot of a robotically assisted surgical system for facilitating predictive maintenance decisions.

FIG. 5 is a diagram illustrating example output actions associated with predictive maintenance decisions of a robotically assisted surgical system.

### SUMMARY

1. A robotically assisted surgical system determines predictive maintenance actions using machine learning techniques. Input data is obtained (which may include operational data, kinematics data, or other information) that are associated with operation of the robotically assisted surgical system. A machine learning model is applied to the input data to predict a likelihood of a future failure event that may occur in an absence of a maintenance action. The robotically assisted surgical system determines if the likelihood meets an action threshold for taking an action relaying to predictive maintenance. Responsive to the likelihood meeting the action threshold, the robotically assisted surgical system determines action data indicative of a preventative maintenance action predicted to counteract the future failure event, and outputs the action data.

In some embodiments, the machine learning model may be trained according to an unsupervised learning approach with respect to historical operations to learn characteristics of anomalous operation. In other embodiments, the machine learning model is trained according to a supervised learning approach to learn relationships between training data (e.g., operational data, kinematics data, etc.) obtained from historical operations and failure events occurring in the historical operations.

In an embodiment, the training and/or input data may relate to sensed operational data include at least one of: a power input to a motor of the robotically assisted surgical system, a rotational velocity of the motor, a linear velocity of a component of the robotically assisted surgical system, a displacement of the component of the robotically assisted surgical system, a force applied by the component of the robotically assisted surgical system, a count of brake actuations, an error code issued by the robotically assisted surgical system, a fault rate associated with the robotically assisted surgical system, a log file associated with the robotically assisted surgical system. The set of operational data may furthermore comprise at least one time-based data series representing a monitored parameter value over a time period.

In various instances, generating the action data may comprise, for example, outputting a notification for display on an output device, outputting an application programming interface (API) message to trigger an action in a platform connected to the robot, and/or initiating an automated remedial action associated with the robot.

In further embodiments, a non-transitory computer-readable storage medium stores instructions executable by a processor for performing any of the methods described above. The methods may be employed in a surgically assisted robotic system including one or more robots and associated electronics for assisting various medical procedures.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

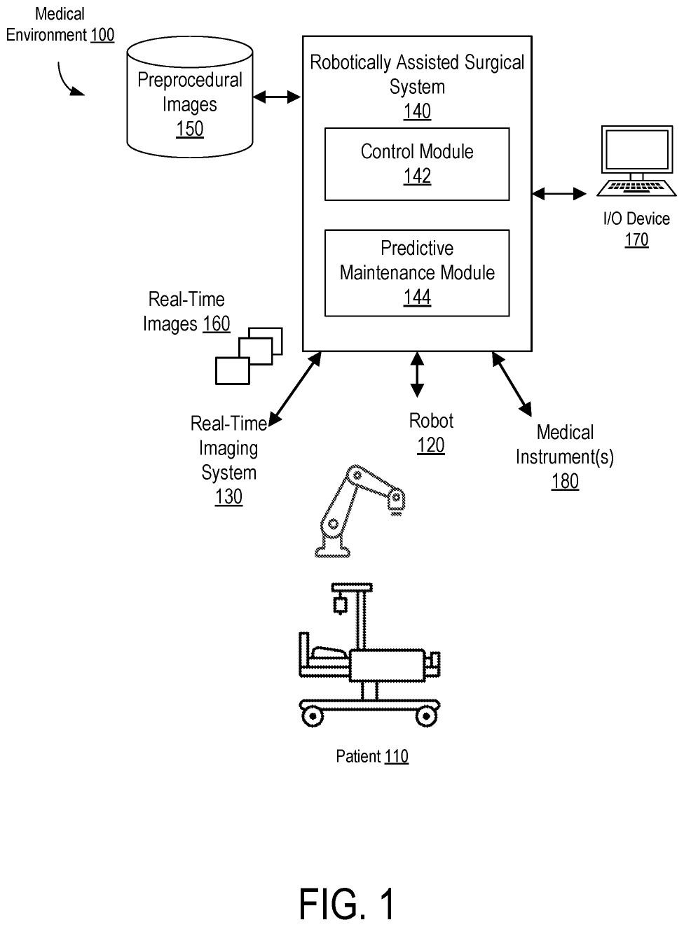

FIG. 1 illustrates an example embodiment of a medical environment 100 associated with a robotically assisted surgical system. The medical environment 100 includes a robotically assisted surgical system 140, a robot 120, a real-time imaging system 130 for capturing real-time images 160, one or more medical instruments 180, a preprocedural image datastore 150 storing preprocedural images, and one or more input/output (I/O) devices 170. In different variations, the medical environment 100 may include different or additional components.

The robotically assisted surgical system 140 and the preprocedural image datastore 150 may be all or partially co-located with the patient 110, the robot 120, real-time imaging system 130, and one or more medical instruments 180 (e.g., in an operating or examination room of a medical facility), or may be at least partially located remotely (e.g., in a server room of a medical facility or in a cloud server system remote from the medical facility). The various electronic components 120, 130, 140, 150, 170, 180 may all or partly be communicatively coupled via one or more networks (e.g., a local area network (LAN), wide area network (WAN), cellular data network, or combination thereof), and/or may be directly communicatively coupled via wired or wireless communication links (e.g., WiFi direct, Bluetooth, Universal Serial Bus (USB), or other communication link).

The preprocedural images 150 comprise a set of images of the patient 110 captured prior to a medical procedure. The preprocedural images 150 may comprise, for example, CT scan images, magnetic resonance imaging (MRI) images, ultrasound images, X-ray images, positron emission tomography (PET) images, or other images or combinations thereof relevant to the procedure. The preprocedural images 150 may be annotated to indicate one or more target locations associated with an anatomical target (e.g., a tumor or other nodule, mass, lesion, or other anatomical structure) of the procedure.

The real-time imaging system 130 captures real-time images 160 of the patient 110 during the medical procedure. The real-time imaging system 130 may comprise an endoscope having one or more light sources and one or more light sensors (e.g., cameras) coupled to a probe tip of a long, thin, tube (e.g., rigid or flexible) that can be threaded through various anatomical channels such as airways, blood vessels, gastrointestinal tract, or other channels, or other pathways (such as through tissue) including those that may be formed by a needle, cannula, or other instrument. The real-time imaging system 130 may furthermore include one or more sensors (e.g., an electromagnetic coil) proximate to the probe tip that enables a location-sensing system to detect a location of the probe tip as it traverses through the anatomy.

In other embodiments, the real-time imaging system 130 may comprise one or more overhead mounted cameras, a head-mounted camera worn by a medical practitioner, or a handheld camera. The real-time imaging system 130 may capture light images or other various types of images such as thermal images, X-ray images, or other images. The real-time imaging system 130 may furthermore comprise a single camera, stereoscopic camera, or multi-view camera.

The medical instrument 180 may comprise an instrument for facilitating a medical procedure. The medical instrument 180 may comprise, for example, a cutting instrument (e.g., a scalpel, scissor, laser, etc.), a suturing or stapling instrument, a sensing instrument for sensing various conditions, a medicant delivery instrument (e.g., comprising a delivery needle and a medicant delivery pump), a biopsy instrument for obtaining a tissue sample, or other instrument associated with a medical procedure. The medical instrument 180 may optionally include integrated electronics for receiving control signals and/or sending sensing data to the robotically assisted surgical system 140.

In some embodiments, the medical instrument 180 may physically couple and/or electronically interface with the real-time imaging system 130. For example, an endoscope of the real-time imaging system 130 could also have one or more working channels that enable one or more instruments 180, such as a needle, scalpel, probe, or other instrument 180, to pass through the endoscope to a position proximate to the camera. In some embodiments, the real-time imaging system 130 and one or more medical instruments 180 may be integrated into a single device.

The robot 120 may facilitate movement of the real-time imaging system 130 and/or the one or more medical instruments 180 through one or more anatomical channels or other pathways of the patient 110 (e.g., through tissue) to a target location. The robot 120 may comprise for example, a robot arm or other electronically controlled device with an attachment point for attaching to the real-time imaging system 130, the one or more medical instruments 180 or both. The robot 120 may furthermore facilitate actions of a medical instrument 180 such as dispensing medicant, extending a needle for biopsy collection, powering a laser, etc.

In different embodiments, the robot 120 may operate with different levels of autonomy. For example, in some embodiments, the robot 120 is entirely under control of a human operator that controls movements using the I/O device 170. In this embodiment, the robot 120 may be controlled responsive to control inputs provided by a medical professional using a I/O device 170 (e.g., a handheld controller or other computer input device). Alternatively, the robot 120 may be controlled in an autonomous way based on autonomous navigation commands generated by the robotically assisted surgical system 140. The automated commands may be based on, for example, a preplanned procedure, feedback from the real-time imaging system 130 or other sensing probes, or other input signals. In further embodiments, the robot 120 may be controlled based on a combination of manual inputs from a medical professional and autonomous signals. For example, a medical professional may initiate certain preplanned movements using a controller, and the robot 120 may automatically perform one or more preprogrammed control movements responsive to the control inputs.

In some embodiments, the robot 120 may be interchangeably adapted for use with different real-time imaging systems 130, medical instruments 180, or a combination thereof. Alternatively, a robot 120 may include one or more integrated imaging systems 130, medical instruments 180 or combinations thereof.

As will be further explained below, the robot 120 may include various components such as one or more motors, one or more actuators, various sensors, and various control and sensing electronics. The robot 120 may furthermore include various fastening elements (e.g., screws, bolts, nuts, etc.), various sealing components (e.g., gaskets or other seals), various mechanical components (e.g., gears, shafts, pins, etc.), various structural components (e.g., housings, covers, panels, etc.), or other parts. Some such parts may be prone to wearing out or failing over time. Furthermore, various parts may be prone to unexpected failures or defects (which may occur during early life) due to manufacturing anomalies, environmental conditions, atypical usage patterns, or other conditions. Further still, failures may occur for unknown or seemingly random reasons during the normal lifespan due to intrinsic unreliability of components. As will further be described below, the robotically assisted surgical system 140 may monitor operation of the robot 120 and generate inferences to facilitate predictive maintenance, thereby reducing the likelihood of failures or degradation of components.

The I/O device 170 may render various views of the preprocedural images 150 and/or the real-time images 160 to facilitate the medical procedure. Various virtual objects and/or other data may optionally be overlaid on the preprocedural images 150 and/or the real-time images 160. For example, a location of the target marked in the preprocedural images and registered to the real-time images 160 may be rendered as a virtual object in a display of the real-time images 160. Furthermore, a tracked location of the probe tip of the real-time imaging system 130 or a probe tip of a medical instrument 180 may be mapped to coordinates in the preprocedural images 150 and overlay on the preprocedural images 150 to track movement of the real-time imaging system 130 and/or the medical instrument 180.

The robotically assisted surgical system 140 comprises a robot control module 142 and a predictive maintenance module 144. The robot control module 142 enables real-time tracking of the real-time imaging system 130 and/or medical instruments 180 using electromagnetic tracking, image-based tracking (including white light and/or fluorescent light tracking), shape sensing, or other techniques. The robot control module 142 may furthermore control actuation of certain types of medical instruments 180 such as, for example, activating a medicant delivery pump of a medicant delivery instrument, actuating a cutting action of an electronic scalpel, actuating a biopsy needle to obtain a tissue sample, etc.

The robot control module 142 may perform a registration between the set of preprocedural images 150 and the real-time images 160 to map between respective coordinates of the image spaces in the preprocedural images 150 and the real-time images 160. In this way, the real-time position of the probe end of the real-time imaging system 130 can be mapped to a specific position in the image space of the preprocedural images 150. Furthermore, the target position of the anatomical target in the preprocedural images can be mapped to a virtual target location in the real-time images. A location of sensing tip of a medical instrument 180 may similarly be registered to the real-time images 160 and/or the preprocedural images based on predefined physical alignment between the medical instrument 180 and the probe tip of the real-time imaging system 130, electromagnetic tracking, image-based tracking, or a combination thereof.

The robot control module 142 may furthermore determine a navigation path to a target position in the preprocedural images 150. For example, the robot control module 142 may generate control commands for controlling navigation of the real-time imaging system 130 and/or the medical instrument 180 to a vicinity of the target.

The predictive maintenance module 144 infers a system health of the robot 120 and predicts likelihoods of future failures associated with the robot 120 in the absence of maintenance actions. The prediction maintenance module 144 may then issue alerts with recommended actions for reducing or eliminating the likelihood of failure. For example, the predictive maintenance module 144 may issue alerts recommending replacing a part, testing a part, performing a calibration associated with one or more parts, or performing a maintenance action on a part (e.g., cleaning, lubricating, tightening, etc.). The predictive maintenance module 144 may furthermore determine various parameters associated with the recommended actions such as a severity level, a targeted alert recipient (e.g., the surgeon, a scheduler, a robotic technician, etc.), and a confidence level associated with the recommended action. In some embodiments, the predictive maintenance module 144 may initiate automated actions such as, for example, performing a whole system or sub-system reset, performing a recalibration, initiating a diagnostic test, etc. The predictive maintenance module 144 may therefore serve as an early warning detection system that may reduce or eliminate unanticipated degradations or failures of the robot 120.

The predictive maintenance module 144 may determine recommended actions based in part on one or more machine learning models trained on historical operational data associated with the robot 120. For example, the predictive maintenance module 144 may employ a machine learning model trained to predict occurrences of specific failures (or classes of failures) based on historical observed failures and the operational data leading up to those failures. Alternatively, the machine learning model may be trained based on actual or suggested maintenance activities taken or recommended by a technician without the failure actually occurring. In other embodiments, the machine learning model may be trained to detect anomalies in the observed operating data based on historical operating data of robots 120 (without failures necessarily occurring). Here, different ML models may be trained in association with different medical procedures (or classes of different medical procedures) to enable detection of operational anomalies that may be specific to a certain procedure.

The robotically assisted surgical system 140 may be implemented using on-site computing and/or storage systems, cloud computing and/or storage systems, or a combination thereof. Accordingly, the robotically assisted surgical system 140 may be local, remote, and/or distributed with portions being local and portions remote, where the various system elements may be communicatively coupled over a network. The robotically assisted surgical system 140 may implement the functions described herein by one or more processor and a non-transitory computer-readable storage medium that stores instructions executable by the one or more processors to perform the described functions.

In other embodiments, the robotically assisted surgical system 140 may operate without necessarily utilizing preprocedural images from the preprocedural image database 150 and may instead operate based only on the real-time images 160. In further embodiments, the robotically assisted surgical system 140 may operate without necessarily relying on real-time images 160, e.g., for procedures where the surgeon has a direct line-of-sight to an anatomical target of the procedure.

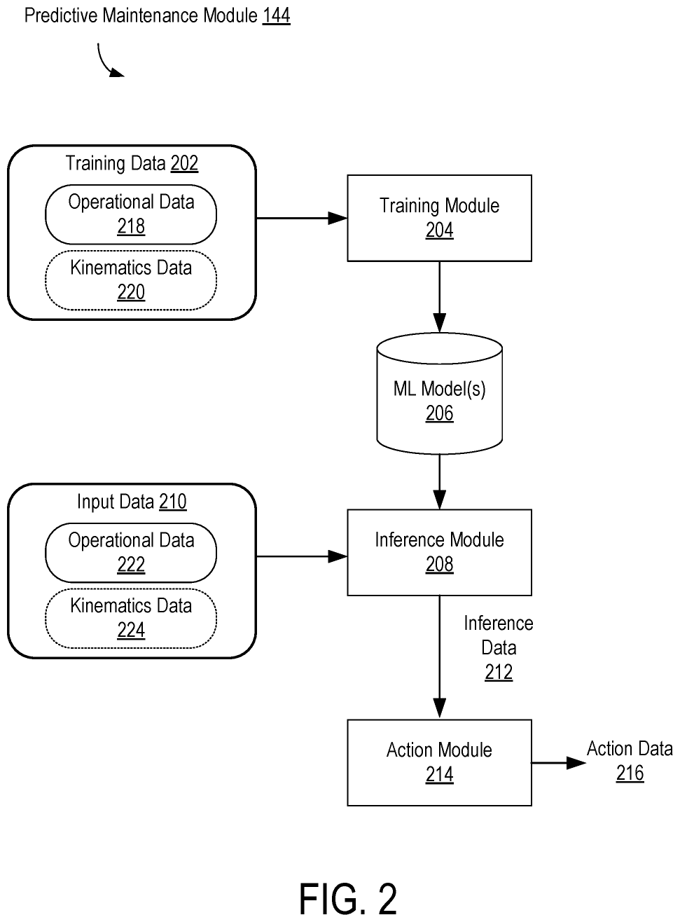

FIG. 2 is an example embodiment of a predictive maintenance module 144 that operates in conjunction with a robotically assisted surgical system 140. The predictive maintenance module 144 includes a training module 204 for training one or more machine learning (ML) models 206 based on training data 202 (e.g., operational data 218 and optionally robot kinematics data 220), an inference module 208 that applies the one or more ML models 206 to generate inference data 212 relating to predictive maintenance based on input data 210 (e.g., operational data 222 and optionally robot kinematics data 224), and an action module 214 for generating one or more action outputs 216 based on the inference data 212.

In various embodiments, the training input data may generally be derived from input commands received by the robot, state information associated with operational and/or kinematic states of the robot, sensed data associated with operations, training or surgery videos or other media associated with the robot, or other types of data. The training input data 202 may be in the form of numerical data (which may represent certain commands, states, etc.), textual data (e.g., operational logs), or multimedia data (e.g., images, video, animations, etc.). Examples of

The training operational data 218 may comprise historical data associated with operation of one or more robots 120 in actual or simulated performances of medical procedures. The training operational data 218 may relate to control commands that control operation of the robot 120, sensing data representing sensed conditions associated with operation of the robot 120, and/or fault data indicative of faults generated by the robot 120. Examples of control commands may include, for examples, commands controlling motion of the robot 120 (e.g., velocity, displacement, acceleration, force, etc.) or actuation commands associated with actuating an actuator of the robot 120 or connected instrument 180. The motion control commands may relate to control parameters associated with motion of an instrument 180 or drive parameters associated with operation of a driving motor (e.g., power delivered to a motor, rotation rate, torque, etc.). The sensed conditions may comprise sensed motion conditions relating to an attached instrument 180 or the motors (e.g., displacement, velocity, acceleration, etc.) or other sensed conditions such as temperature, applied torque, pressure, etc. relating to operations of the robot 120. The fault conditions may relate to various error conditions generated by the robot 120 which may relate to communication errors, power delivery errors, control response errors, etc. Fault conditions may be designated by predefined error codes specific to the robot 120 or its respective sub-systems.

The training operational data 218 may furthermore include indications of failures of one or more components of the robot 120. Failures may comprise manually or automatically notated failures and may include information about the specific failed component, the source of failure, or other information pertaining to the failure. Failures may relate to physical elements such as seals, fastening structures, hinges, motors, shafts, actuators, etc., electronic components such as capacitors, transducers, power supplies, etc., communication systems, or control modules. Failures may furthermore relate to firmware or software components and may be indicative of software bugs, incompatibilities, security issues, or outdated software. Alternatively, or in addition, the training operational data 218 may include indications of maintenance actions performed by a technician. Such actions may be indicative of an anticipated failure by a trained technician and/or best practices to avoid failure without an actual failure occurring. The failures in the training operation data 218 may furthermore include detected or anticipated degradation of components that may not necessarily by itself prevent continued operation of the robot 120.

At least some of the training operational data 218 may comprise time series data that indicate a series of values over a historical time frame. Furthermore, the failure data may indicate relative timing of the occurrence or predicted occurrence of a failure. The training operation data 202 may comprise raw values (e.g., from system logs or other data sources) and/or may comprise filtered, normalized, or converted values. For example, motion parameter values may be converted to error values indicative of differences between an input motion control input and an actual measured motion. Furthermore, some values may be converted from numerical values to binary values indicative of whether or not the numerical values are above or below a predefined threshold. In further embodiments, various feature extraction or other pre-processing techniques may be applied to a set of raw data to generate the training operational data 202.

The robot kinematics data 220 may describe various structural design aspects of the robot 120. For example, the robot kinematics data 220 may describe relationships between the dimensions and connectivity of various kinematic chains of the robot 120 and the position, velocity and acceleration of each of the links in the robot 120.

The training module 204 applies one or more machine learning algorithms to the training data 202. In one embodiment, the training module 204 employs a supervised learning technique to learn relationships between the training data 202 and the failures, degradations, and/or maintenance activities that are observed or predicted by a technician. Here, the training data 202 may be aggregated into time series feature vectors representing varying operational data over time, which are labeled according to the observed failures. The labels may include a relative timing of the observed or predicted failure (e.g., in one week, in 3 months, in 6 months, etc.). The training module 204 may then learn model parameters that enable inferences of failure likelihoods from input operational data. In an embodiment, training module 204 may express the likelihoods in a time series indicative of failure likelihoods over different future time ranges. In an embodiment, the training module 204 may train multiple ML models 206 for inferring different types of failures or timing of failures. Alternatively, a single ML model 206 may produce multiple outputs indicating respective likelihoods of different types of failures or timing of failures.

The training module 204 may alternatively, or additionally, apply one or more unsupervised machine learning algorithms to train an ML model 206 for detecting anomalies. For example, the training module 204 may perform clustering of the training data 202 to learn characteristics of non-anomalous operational states and enable identification of outliers that may be indicative of anomalous operation. An anomalous operational state may be indicative of an increased likelihood of failure even in the absence of specific occurrences of failures in the training data 202. In an embodiment, the training module 204 may generate multiple ML models 206 associated with different medical procedures to enable anomaly detection with respect to a specific planned procedure or class of procedures.

In further embodiments, the training module 204 may be trained according to various classification techniques to classify an operational state of the robot 120. Here, the training module 204 may learn classifications between one or more healthy states of the robot 120, one or more known failure modes of the robot 120, and one or more unknown anomalous states of the robot 120. The different healthy states may correspond to states with different expected operational data that may vary based on different configurations of the robot 120 or may vary based on the facility where the robot 120 is deployed, the surgeon employing the robot 120, the medical procedure being performed or other variables. Different failure modes may relate to different types of known failure and may also vary depending on operational variables described above. The unknown anomalous states may correspond to states that do not correspond to known healthy states or known failure modes, but may nevertheless be indicative of potential failure or degradation.

In various embodiments, the training module 204 may employ machine learning techniques such as neural networks, linear models, tree-based regression, support vector machines (SVM), gradient boosting regression or other regression techniques, clustering, classification, or other machine learning techniques capable of achieving the functions described herein. The training module 204 may furthermore employ one or more large language models (LLMs) such a Generative Pre-trained Transformer (GPT), Language Models for Dialogue Applications (LaMDA, Bard model, for the purpose of preprocessing training data 202 and/or directly incorporating into the one or more ML models 206. Such LLMs may be utilized for tasks such as incorporating and/or interpreting domain-specific data, classifying events, and making inferences.

The inference module 208 applies the one or more ML models 206 to generate inference data 212 relating to potential failures of the robot 120 based on input data 210. The input data 210 may comprise similar data to the training data 202 discussed above and may include operational data 222, and optionally robot kinematics data 224.

The inference module 208 may generate the inference data 212 as one or more likelihoods of a predicted failure in the absence of one or more maintenance activities being performed. Here, the likelihoods may comprise multiple values associated with different types of possible failures and/or may include likelihoods associated with different future time periods (e.g., in the next two weeks, in the next two months, etc.). In the embodiment, the inference module 208 may furthermore generate confidence values associated with the various likelihood predictions. The inference module 208 may furthermore generate inference data 212 indicative of a classification of an operational state of the robot 120. For example, the inference module 208 may classify the state as a healthy state, a known failure mode, or an anomalous state. The inference module 208 may output the inference data 212 as likelihood values and/or confidence values associated with an anomaly or classified state of the robot 120. The inference module 208 may employ similar machine learning algorithms as described above.

The action module 214 generates one or more action outputs 216 based on the inference data 212 from the inference module 208. The action outputs 216 may comprise, for example, notifications sent to an I/O device 170 to alert a medical professional, maintenance technician, scheduler, or other personnel of the predictions. Notifications may furthermore comprise application programming interface (API) calls to a maintenance platform, surgical scheduling platform, or other platform connected to the robotically assisted surgical system 140. In an embodiment, the action module 214 may selectively send different notifications to different personnel and/or integrated platforms depending on the inference data 212. For example, inferences that indicate a sufficiently high likelihood of an imminent failure during a procedure may be sent to an I/O device 170 in the operating room to alert a surgeon or other medical professional. Other inferences may be indicative of a non-imminent recommended maintenance task. Such information may be sent to a scheduler to enable scheduling of downtime associated with repair or maintenance activities. Notifications may also be sent to a device associated with a technician for performing service on the robot 120. In some instances, an inference may result in a recommendation to perform maintenance earlier than an otherwise scheduled maintenance action (e.g., as may be recommended by a manufacturer). In other instances, an inference may indicate that regularly scheduled maintenance is not necessarily needed, and may recommend delaying a maintenance action to a time later than otherwise scheduled maintenance. In some instances, when inferences are available with sufficient confidence, the action module 214 may rely solely on performing “on-demand” maintenance as predicted by the inference module 208 in place of a regularly scheduled maintenance plan. Such on-demand maintenance may furthermore be coordinated with other activities to schedule maintenance at a convenient time, such as at night when the operating room is not being used, or in coordination with other robots 120 that may be rotated in and out of service.

In some embodiments, the action module 214 may automatically invoke one or more actions responsive to inferences from the inference module 208. For example, in response to certain types of inference data 212, the action module 214 may automatically initiate tasks such as performing a calibration, performing a system or sub-system reset, performing a diagnostic test, or initiating an automated maintenance cycle.

In an embodiment, the action module 214 may employ a rule-based approach to processing inference data 212 to generate the action data 216. Rules may be customer-programmable to enable different preferences associated with alert generation and automation of actions. In another embodiment, the action module 214 may apply a separate machine learning model trained to determine specific alerts or actions responsive to the inference data 212 generated by the inference module 208. Here, the machine learning model may be trained based on actual actions or feedback received responsive to notifications.

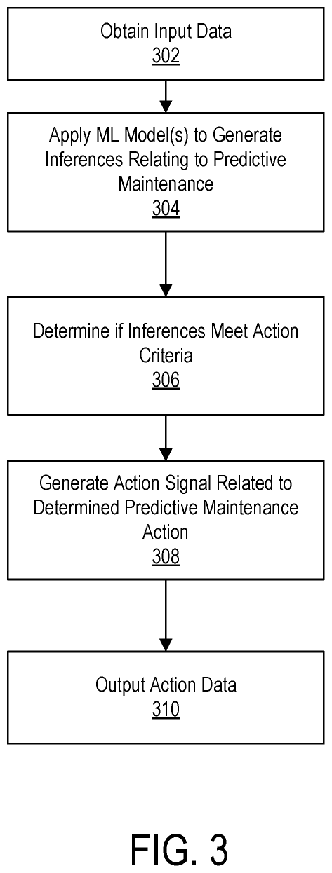

FIG. 3 illustrates an example embodiment of a process for predicting maintenance activities in a robotically assisted surgical system. The predictive maintenance module 144 obtains 302 a set of input data 210 (including operational data 222 and optionally robot kinematics data 224) associated with operation of the robot 120. The predictive maintenance module 144 applies 304 one or more ML models to the input data 210 to predict a likelihood of a future failure event in an absence of a maintenance action. Here, the predictive maintenance module 144 may train the ML model according to a supervised learning approach to learn relationships between a set of training operational data obtained from historical operations and failure events occurring in the historical operations. In another embodiment, the ML model is trained according to an unsupervised learning approach with respect to historical operations to learn characteristics of anomalous operation. The operational data may comprise least one of: a power input to a motor of the robotically assisted surgical system, a rotational velocity of the motor, a linear velocity of a component of the robotically assisted surgical system, a displacement of the component of the robotically assisted surgical system, a force applied by the component of the robotically assisted surgical system, a count of brake actuations, an error code issued by the robotically assisted surgical system, a fault rate associated with the robotically assisted surgical system, a log file associated with the robotically assisted surgical system. The operational data may include time series data representing a monitored parameter value over a time period.

The predictive maintenance module 144 determines 306 if the likelihood meets action criteria for initiating an action. Responsive to the likelihoods meeting the action criteria, the predictive maintenance module 144 generates 308 an action output relating to a preventative maintenance action item predicted to counteract the future failure event. The predictive maintenance module 144 outputs 310 the action. For example, the predictive maintenance module 144 may output a notification to an output device. Furthermore, in some embodiments, the predictive maintenance module 144 may automatically initiate one or more actions such as performing a calibration, executing a diagnostic test, or performing a system or sub-system reset.

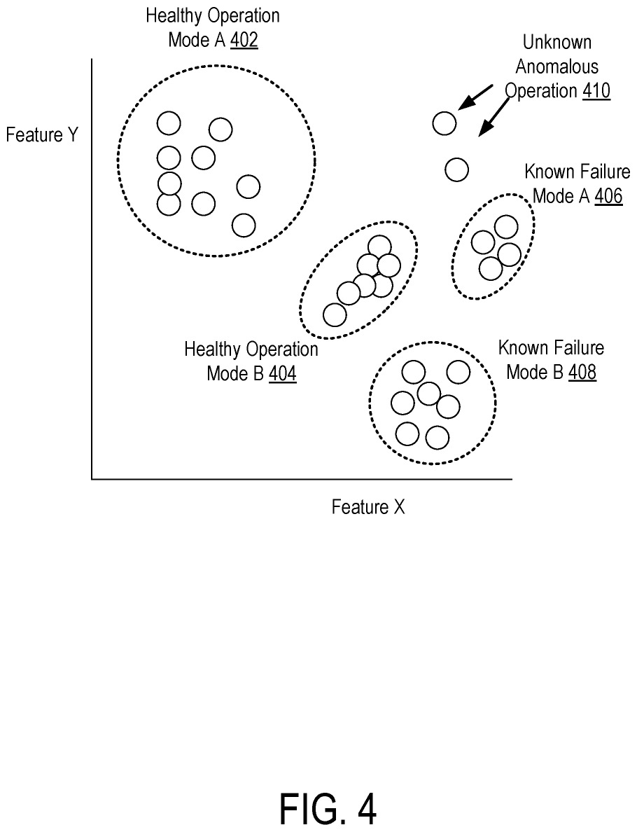

FIG. 4 is a graph conceptually illustrating an operating technique associated with the predictive maintenance module 144. For illustrative purposes only two axes (corresponding to features X and Y) are illustrated in FIG. 4. In practice, the predictive maintenance module 144 may operate based on highly multi-dimensional data that may be based on tens, hundreds, or thousands of input features. Furthermore, while FIG. 4 shows a highly simplified graph associated with an single time snapshot, a practical implementation of the ML model 206 may generate inferences based on time-series data covering a potentially significant historical time window.

In the illustrated example, each plotted point represents a particular combination of observed operational data that define an operational state. Spatially close data points represent similar operational states that will be similarly classified by the ML model 206. In this example, the ML model 206 learns a set of classifications that may include one or more states indicative of “healthy” operation (e.g., healthy operation mode A 402 or healthy operation mode B 404), one or more states indicative of known failure modes (e.g., known failure mode A 406 or known failure mode B 408), and one or more states indicative of unknown anomalous states 410. The different healthy states 402, 404 may correspond to different operating modes of the robot 120 that may result in different expected operational data. For example, different healthy states 402, 404 may correspond to healthy operation associated with different configuration states, different surgeons or other operators of the robot 120, different facilities, different medical procedures, or other variations that may be expected to result in different operational data. The known failure modes 406, 408 may correspond to different types of expected failures or degradations. For example, the different known failure modes 406, 408 may correspond to predicted failures or degradations associated with different components of the robot 120, different expected time to failure, different urgency levels, or failures while operating under different operation conditions resulting from different system configurations, different surgeons or operators of the robot 120, different facilities, different medical procedures, or other variations. The unknown anomalous state 410 corresponds to observed operational data that are not classified as “healthy” but also do not correspond to an existing known failure mode. These states may implicitly indicate that maintenance or further diagnostics are advisable even though it may not predict a specific expected failure or timing of the failure.

While FIG. 4 is limited to only a small number of example classifications for illustrative purposes, a practical implementation may include training one more ML models 206 capable of classifying input data between any number of healthy operational modes, known failure states, and/or anomalous states. The action module 214 may initiate different actions depending on the classification. For example, different classifications may variably generate notifications for different personnel or connected platforms or may variably initiate automated actions dependent on the classification.

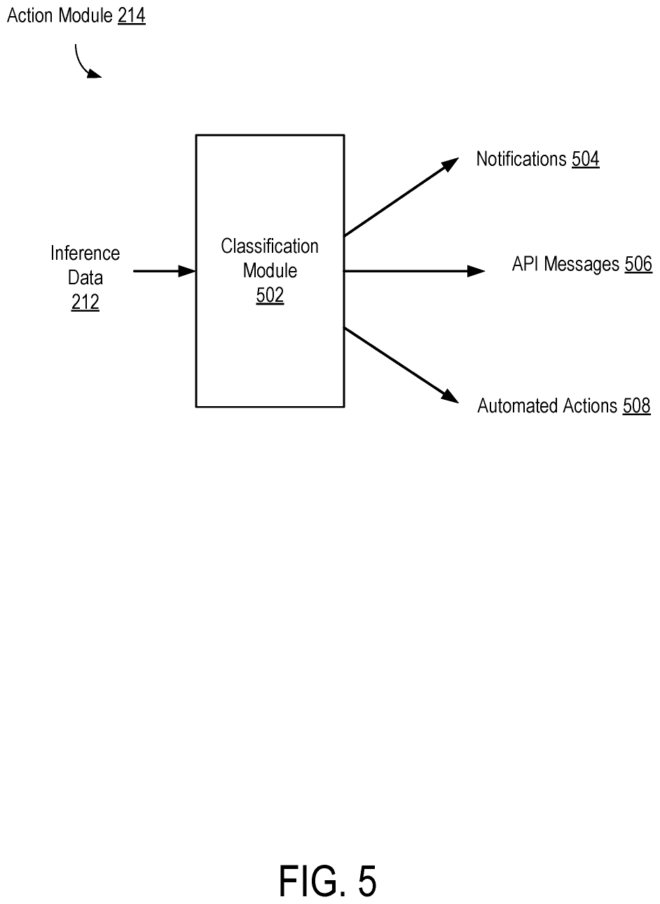

FIG. 5 is a diagram illustrating an example operation of the action module 214 to invoke various types of action outputs 216. In this example, the action module 214 includes a classification module 502 that analyzes the inference data 212 and determines one or more types of actions based on the classifications. Here, the classification module 502 may determine, for example, one or more components implicated in a predicted failure, a severity level (e.g., how significantly the failure would affect operation of the robot 120), an urgency classification (e.g., based on time to failure and/or severity) a recommended action associated with the prediction, a downtime associated with performing a maintenance activity, a notification target, etc. Depending on the classification, the classification module 502 may output one or more notifications 504 to an I/O device, one or more API messages 506 to a connected server, or initiate one or more automated remediation actions 508.

Notifications 504 may be configured based on customer preferences to send varying notifications with varying levels of information to different personnel. For example, for critical failures that may impact an ongoing medical procedure, a notification 504 may be sent to an I/O device in the operating room to alert the medical staff. For recommended maintenance activities that may affect future scheduling of procedures, the notification 504 may be sent to an I/O device of a scheduling staff member together with an estimate of anticipated downtime. More detailed information relating to the inference data 212 may be sent to an I/O device of a technician tasked with servicing the robot 120.

The API messages 506 may be automatically sent to various platforms that may integrate with the robotically assisted surgical system 140. For example, an API message 506 may be sent to a scheduling platform to automatically calendar maintenance activities. Here, API messages 506 may correspond to “on-demand” recommended maintenance (derived from the inferences), which may supplement a regularly scheduled maintenance plan (e.g., by recommending that maintenance be performed earlier or later than the next regularly scheduled maintenance), or may replace a regularly scheduled maintenance plan. API messages 506 furthermore be sent to an ordering system, to trigger orders associated with parts that may require replacement. Messages may also be communicated or logged internally to the robotically assisted surgical system 140 to affect operation of the robot 120.

The automated remediation actions 508 may include tasks such as, for example, automatically performing a system or sub-system reset of the robot 120, automatically performing a recalibration of one or more components of the robot 120, automatically enabling or disabling a component of the robot 120, automatically performing a software or firmware update of the robot 120, or initiating another action that improves performance of the robot 120 based on the inference data 212.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for predicting maintenance activities in a robotically assisted surgical system, the method comprising:
obtaining operational data associated with operation of the robotically assisted surgical system;
applying a machine learning model to the operational data to predict a likelihood of a future failure event in an absence of a maintenance action;
determining if the likelihood meets an action threshold;
responsive to the likelihood meeting the action threshold, generating action data indicative of a preventative maintenance action item predicted to counteract the future failure event; and
outputting the action data.

2. The method of claim 1, wherein the machine learning model is trained according to an unsupervised learning approach with respect to historical operations to learn characteristics of anomalous operation.

3. The method of claim 1, wherein the machine learning model is trained according to a supervised learning approach to learn relationships between a set of training operational data obtained from historical operations and failure events occurring in the historical operations.

4. The method of claim 1, wherein the operational data include at least one of: a power input to a motor of the robotically assisted surgical system, a rotational velocity of the motor, a linear velocity of a component of the robotically assisted surgical system, a displacement of the component of the robotically assisted surgical system, a force applied by the component of the robotically assisted surgical system, a count of brake actuations, an error code issued by the robotically assisted surgical system, a fault rate associated with the robotically assisted surgical system, a log file associated with the robotically assisted surgical system.

5. The method of claim 1, wherein the operational data comprise at least one time-based data series representing a monitored parameter value over a time period.

6. The method of claim 1, wherein generating the action data comprises outputting a notification for display on an output device.

7. The method of claim 1, wherein generating the action data comprises outputting an application programming interface (API) message to trigger an action in a platform connected to the robotically assisted surgical system.

8. The method of claim 1, wherein generating the action data comprises initiating an automated remedial action associated with the robotically assisted surgical system.

9. The method of claim 1, wherein generating the action data comprises recommending an on-demand maintenance activity independent of a scheduled maintenance plan.

10. A non-transitory computer-readable storage medium storing instructions for predicting maintenance activities in a robotically assisted surgical system, the instructions when executed by a processor causing the processor to perform steps including:
obtaining operational data associated with operation of the robotically assisted surgical system;
applying a machine learning model to the operational data to predict a likelihood of a future failure event in an absence of a maintenance action;
determining if the likelihood meets an action threshold;
responsive to the likelihood meeting the action threshold, generating action data indicative of a preventative maintenance action item predicted to counteract the future failure event; and
outputting the action data.

11. The non-transitory computer-readable storage medium of claim 10, wherein the machine learning model is trained according to an unsupervised learning approach with respect to historical operations to learn characteristics of anomalous operation.

12. The non-transitory computer-readable storage medium of claim 10, wherein the machine learning model is trained according to a supervised learning approach to learn relationships between training operational data obtained from historical operations and failure events occurring in the historical operations.

13. The non-transitory computer-readable storage medium of claim 10, wherein the operational data include at least one of: a power input to a motor of the robotically assisted surgical system, a rotational velocity of the motor, a linear velocity of a component of the robotically assisted surgical system, a displacement of the component of the robotically assisted surgical system, a force applied by the component of the robotically assisted surgical system, a count of brake actuations, an error code issued by the robotically assisted surgical system, a fault rate associated with the robotically assisted surgical system, a log file associated with the robotically assisted surgical system.

14. The non-transitory computer-readable storage medium of claim 10, wherein the operational data comprise at least one time-based data series representing a monitored parameter value over a time period.

15. The non-transitory computer-readable storage medium of claim 10, wherein generating the action data comprises outputting a notification for display on an output device.

16. The non-transitory computer-readable storage medium of claim 10, wherein generating the action data comprises outputting an application programming interface (API) message to trigger an action in a platform connected to the robotically assisted surgical system.

17. The non-transitory computer-readable storage medium of claim 10, wherein generating the action data comprises initiating an automated remedial action associated with the robotically assisted surgical system.

18. A robotically assisted surgical system comprising:
a robot for facilitating assistance associated with a medical procedure;
a processor; and
a non-transitory computer-readable storage medium storing instructions for predicting maintenance activities in a robotically assisted surgical system, the instructions when executed by the processor causing the processor to perform steps including:
obtaining operational data associated with operation of the robot;
applying a machine learning model to the operational data to predict a likelihood of a future failure event in an absence of a maintenance action;
determining if the likelihood meets an action threshold;
responsive to the likelihood meeting the action threshold, generating action data indicative of a preventative maintenance action item predicted to counteract the future failure event; and
outputting the action data.

19. The robotically assisted surgical system of claim 18, wherein the machine learning model is trained according to an unsupervised learning approach with respect to historical operations to learn characteristics of anomalous operation.

20. The robotically assisted surgical system of claim 18, wherein the machine learning model is trained according to a supervised learning approach to learn relationships between training operational data obtained from historical operations and failure events occurring in the historical operations.
